(19) United States
(12) Patent Application Publication
(10) Pub. No.: US 2011/0043759 A1
(43) Pub. Date: Feb. 24, 2011

(54) METHOD AND SYSTEM FOR DETERMINING FAMILIARITY WITH STIMULI

(75) Inventor: Shay BUSHINSKY, Ganei-Tikva (IL)

Correspondence Address: Fleit Gibbons Gutman Bongini & Bianco PL, 21355 EAST DIXIE HIGHWAY, SUITE 115, MIAMI, FL, 33180, US

(73) Assignees: Shay Bushinsky, Ganei-Tikva (IL); ATLAS INVEST HOLDINGS LTD., Tortola (VG)

(21) Appl. No.: 12/886,158

(22) Filed: Sep. 20, 2010

Related U.S. Application Data
(63) Continuation-in-part of application No. PCT/IL2009/000308, filed on Mar. 18, 2009.
(60) Provisional application No. 61/037,332, filed on Mar. 18, 2008.

(51) Int. Cl. A61B 3/113

(52) U.S. Cl. 351/210; 351/246

(57) ABSTRACT

The present invention comprises a system and method for determining the familiarity of a subject with a given stimulus. The method is based on tracking eye movements of the subject when they are presented with these stimuli, for example by use of an eye-tracking camera adapted for this purpose. Differences in familiarity with a given stimulus will evoke different responses in subjects eye movements, and these differences are analyzed by a classification algorithm in order to determine familiarity with a given stimulus or lack thereof.

### CROSS-REFERENCE TO RELATED APPLICATIONS

This application is a Continuation In Part of PCT International Application No. PCT/IL2009/000308, International Filing Date Mar. 18, 2009, claiming priority of Provisional Patent Application 61/037,332, filed Mar. 18, 2008.

### FIELD OF THE INVENTION

The present invention relates to a method and system for correlating eye movements with mental states, useful for instance for detecting lies.

### BACKGROUND OF THE INVENTION

A variety of systems for lie detection have been devised. Those systems based on physical measurement generally utilize bodily responses not easily controlled, but known to be affected by mental states (such as increased stress). The common polygraph measures changes in skin conductivity due to changes in perspiration levels. Voice stress analysis has also been widely used. Heart rate, blood pressure, functional MRI, electroencephalography, cognitive chronography, and cranial blood flow changes (e.g. using functional transcranial Doppler measurements) have all likewise been used in attempts to detect whether a subject is lying or not. Some key problems in the field are system cost, portability, accuracy of results (i.e. high rates of false readings), and subjectivity of results (as many systems rely on human interpretation of data). Countermeasures such as tongue biting, toe curling, sphincter tightening, or mental manipulation of numbers can often be used effectively against standard polygraphs.

In the patent literature one finds a number of systems purporting to solve one or more of these problems. For example, the ‘intelligent deception verification system’ (U.S. patent Ser. No. 10/736,490) provides a system using a variety of possible inputs including brainwaves, eye, heart, and muscle activity, skin conductance, body temperature, position, posture, expression, gestures, blood flow, blood volume, respiration, blood pressure, heart rate, and the like. These inputs are measured in conjunction with stimuli presented to the subject. An algorithm controls the stimuli and analyzes the inputs in an attempt to evoke clearly responses from the subject that are classifiable as true or false with high accuracy. This algorithm may utilize neural networks or other methods for classification. The preferred embodiment involves presenting stimuli using an immersive virtual reality system, and sensing input by means of a wearable sensor placement unit. It will be appreciated that there may be need for surreptitious determination of veracity, and therefore a wearable sensor placement unit and immersive virtual reality system, while possibly providing reliable measurements of veracity, are not suitable for surreptitious measurement. It would appear the main thrust of '490 is to provide a platform for researchers and field examiners to create interrogation protocols and perform data analysis on many different signal types for research purposes. It would appear that this patent is written so generally that enablement for a specific novel working lie-detector using the elements of the system would not be clear even to one skilled in the art; consider for example claims 11-14, claiming: “one or more sensor placement units . . . one or more digital signal processing units . . . instructions for sending commands to the virtual reality system to generate one or more stimuli . . . receiving one or more signals . . . and performing spatial-frequency analysis on the data to obtain information regarding the likelihood of deception”. From the extremely general claims and the remainder of the patent specification, the simplest questions such as what stimuli to present and how to analyze the measured signals thereby stimulated, remain woefully under-addressed. In fact it has been shown that all known devices using voice data for lie detection “perform at chance level”, and therefore other methods are necessary. [“Charlatanry in forensic speech science: A problem to be taken seriously”, Erikkson and Lacerda, The Int'l Journal of speech, language, and the law, V14 p 169.]

Recent scientific research established the link between eye movement and mental processes occurring in the human brain. For example, researchers Daniel C. Richardson and Rick Dale of Cornell and Stanford Universities have established that eye movement is a function of image features, and the cognitive processes back in the brain. Other factors identified as influencing eye movement include what the viewer is told, what the viewer answers, what the viewer thinks but does not say, and his emotional state. [Looking To Understand: The Coupling Between Speakers' and Listeners' Eye Movements and Relationship to Discourse Comprehension, Cognitive Science 29 (2005) p. 1045]. When fetching from memory, there is a characteristic constant eye movement in an empty space. A particular eye movement was recorded when trying to solve a hard problem. Significant differences were observed between the tracking of eye movement of subjects who were either knowledgeable of what they saw and those who were unknowledgeable. While the knowledgeable ones generated a more consistent eye movement, the uninformed people tended to gaze and scan the visuals in an inconsistent and disoriented manner. Their eyes would run around all over projected images with much less focusing.

Eye movement also serves as a predictor of the degree of a persons' understanding. The “Eye track 3” system developers [http://poynterextra.org/eyetrack2004/main.htm] studied how people view websites in order to help design them better. Their conclusions were in line with the Dale and Richardson research; they identified a clear correlation between the text's layout, size and alignment to the degree of the readers' comprehension of the issues presented.

U.S. Pat. No. 6,102,870 discloses a method for determining mental states from spatio-temporal eye-tracking data, independent of a-priori knowledge of the objects in the person's visual field. The method is based on a hierarchical analysis using eye-tracker samples, features based thereon such as fixations and facades, eye movement patterns based on the features, and mental states based on the eye movement patterns. The method is adapted for classification into a small set of mental states, not including stress or any other states associated with mendacity. In short, the device has not been designed for use as a lie detector. The classes identifiable by the device include line reading (at least two horizontal saccades to the left or right), reading a block (several lines followed by saccades in the direction opposite to the lines), re-reading/scanning/skimming, thinking (long fixations separated by short saccade spurts), spacing out (same as thinking but over long period of time!), searching, re-acquaintance, and ‘intention to select’ (fixation in area designated as ‘selectable’). US patent application WO 2005/022293 discloses a method for detecting deception or information possessed by a subject. The subject is presented with stimuli and a psychophysiological response to the stimuli is measured and classified. The subject is presented with two types of control questions, the responses to which will form the standards for the classification. The two types of control questions are: 1) irrelevant questions and 2) known relevant questions. The subject is then presented with a critical relevant question (relevant to the crime). The response to the critical relevant question is classified as being in one of two different categories, according to the response similarity to either the known relevant responses or the irrelevant responses. Responses of the subject are measured by sensors attached to the subject's body: EEG sensors that collect EEG data originating in the subject's central nervous system, blood pressure sensor, skin conductance sensor, blood flow sensor and the like. According to another embodiment, the test reveals the presence or absence of information stored in the brain.

There is a need to provide an improved method and system for classifying eye movement data into multiple categories other than two positive and negative categories and to evaluate a level of knowledge of the subject and a type of the knowledge.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which

FIGS. 1A, B present a figure with gaze to the right and to the left;

FIG. 2 presents an eye tracking camera and associated hardware;

FIG. 3 presents a typical portion of eye tracking camera output;

FIG. 4 presents a typical device setup of the current invention including user; computer, and eye tracking camera.

FIG. 5 is a flowchart indicating a method for classifying eye movement data into familiarity categories;

FIGS. 6A, 6B and 6C present diagrams illustrating recorded fixation points;

FIG. 7 is a flowchart of a method for evaluating a familiarity level;

FIG. 8 illustrates a flowchart of a method 800 for detecting a symptomatic behavior of lying.

### SUMMARY OF THE INVENTION

The present invention comprises a system and method for determining the familiarity of a subject with a given stimulus. The method is based on tracking eye movements of the subject when they are presented with these stimuli, for example by use of an eye-tracking camera adapted for this purpose. Differences in familiarity with a given stimulus will evoke different responses in subjects eye movements, and these differences are analyzed by a classification algorithm in order to determine familiarity with a given stimulus or lack thereof.

It is within provision of the invention to provide a method for determining a subject's familiarity with given stimuli comprising steps of:
a. providing an eye-movement detection camera adapted to capture and record eye movement data of said subject;
b. providing a display means, adapted for presentation of said stimuli to said subject,
c. providing a computing platform in communication with said camera, adapted for analyzing said eye movement data;
d. presenting said subject with a series of stimuli;
e. recording the eye movements of said subject by means of said eye-movement detection camera; and,
f. classifying the eye movements of said subject using said eye movement data and said computing platform;
wherein the eye movements of said subject are utilized to classify said subject's responses to said stimuli.

It is a further provision of the invention to provide a method as described above, wherein said platform adapted for presentation of stimuli is the same computing platform adapted for determination of said familiarity category.

It is a further provision of the invention to provide a method as described above wherein said platform adapted for presentation of stimuli is the same computing platform running said classification algorithm.

It is a further provision of the invention to provide a method as described above wherein said classification is into at least one member of a group consisting of: admitted familiarity, admitted unfamiliarity, denied familiarity, and denied unfamiliarity.

It is a further provision of the invention to provide a method as described above wherein said classification is accomplished by means of an algorithm selected from a group consisting of: support vector machine [SVM], decision tree, Bayesian network, neural network, genetic algorithm, expert system, pattern matching algorithm, heuristic algorithm, or combinations thereof.

It is a further provision of the invention to provide a method as described above wherein said algorithm is trained on training data selected from a group consisting of: data gleaned from the population at large; data gleaned from population subsets; and data gleaned from said subject.

It is a further provision of the invention to provide a method as described above wherein said eye movement data is selected from a group consisting of: gaze direction, fixation duration, saccade duration, saccade velocity, head position, head velocity, or combinations thereof.

It is a further provision of the invention to provide a method as described above wherein said stimuli are selected from a group consisting of: images known to be familiar to said subject, images known to be unfamiliar to said subject, images suspected to be familiar to said subject, images suspected to be unfamiliar to said subject, images of persons, images of places, images of things, videos, digital media, persons, objects, auditory information, tactile stimuli, olfactory stimuli, or combinations thereof.

It is a further provision of the invention to provide a method as described above further requesting a response from said subject to said stimuli, selected from a group consisting of: talking about said stimuli, observing said stimuli, writing about said stimuli, or classifying said stimuli.

It is a further provision of the invention to provide a method as described above wherein said display means is selected from a group consisting of: a computer display, projector, photograph, sketch, or drawing.

It is a provision of the invention to provide a system for determining a subject's familiarity with given stimuli consisting of:
a. display means adapted for presentation of said stimuli to said subject,
b. an eye-movement detection camera adapted to capture and record eye movement data of said subject;
c. a computing platform in communication with said camera, adapted for analyzing said eye movement data;
wherein the eye movements of said subject are utilized to classify said subject's responses to said stimuli.

It is a further provision of the invention to provide a method as described above wherein said classification is into the groups: admitted familiarity, admitted unfamiliarity, denied familiarity, or denied unfamiliarity.

It is a further provision of the invention to provide a method as described above wherein said classification is accomplished by means of an algorithm selected from a group consisting of: support vector machine [SVM], decision tree, Bayesian network, neural network, genetic algorithm, expert system, pattern matching algorithm, heuristic algorithms, or combinations thereof.

It is a further provision of the invention to provide a method as described above wherein said algorithm is trained on training data selected from a group consisting of: data gleaned from the population at large; data gleaned from population subsets; or data gleaned from said subject.

It is a further provision of the invention to provide a method as described above wherein said eye movement data is selected from a group consisting of: gaze direction, fixation duration, saccade duration, saccade velocity, head position, head velocity, or combinations thereof.

It is a further provision of the invention to provide a method as described above wherein said stimuli are selected from a group consisting of: images known to be familiar to said subject, images known to be unfamiliar to said subject, images suspected to be familiar to said subject, images suspected to be unfamiliar to said subject, images of persons, images of places, images of things, videos, digital media, persons, objects, auditory information, tactile stimulation, olfactory stimulation, or combinations thereof.

It is a further provision of the invention to provide a method as described above further requesting a response from said subject to said stimuli, selected from a group consisting of: talking about said stimuli, observing said stimuli, writing about said stimuli, or classifying said stimuli.

It is a further provision of the invention to provide a method as described above wherein said display means is selected from a group consisting of: a computer display, projector, photograph, sketch, or drawing.

It is a further provision of the invention to provide a method as described above, wherein said eye movement data comprises position attributes of fixations, and wherein said determining of said familiarity category is based on said position attributes.

It is a further provision of the invention to provide a method as described above wherein the step of determining comprises:
calculating a condensation level of a spatial distribution of said fixations, based on said position attributes; and
evaluating a level of familiarity, wherein said level of familiarity is in a direct proportion to said condensation level.

It is a further provision of the invention to provide a method for detecting symptomatic behavior of lying, comprising steps of:
a. determining a subject's familiarity with given stimuli comprising steps of
   i. providing an eye-movement detection camera adapted to capture and record eye movement data of said subject;
   ii. providing a display means, adapted for presentation of said stimuli to said subject,
   iii. providing a computing platform in communication with said camera, adapted for analyzing said eye movement data;
   iv. presenting said subject with a stimulus;
   v. recording eye movements data associated with said subject's response to said stimulus, by said eye-movement detection camera and said computing platform; and
   vi. determining, based on said eye movement data, a familiarity category selected from multiple familiarity categories, wherein said familiarity category defines a familiarity of said subject with said stimulus and
b. implementing a lying detection technique on said subject and obtaining a lying detection result therefrom
c. combining said lying detection result with said determined familiarity category such that an overall detection quality result is obtained.

It is a further provision of the invention to provide the aforementioned method wherein said detection quality result has a more than additive accuracy of detection relative to the accuracy of detection obtained from either determining a subject's familiarity with given stimuli or implementing a lying detection technique on said subject and obtaining a lying detection result therefrom alone.

It is a further provision of the invention to provide a system for detecting symptomatic behavior of lying comprising
a. a system for determining a subject's familiarity SSF with given stimuli consisting of:
   i. display means adapted for presentation of said stimuli to said subject,
   ii. an eye-movement detection camera adapted to capture and record eye movement data, associated with said subject's response to a stimulus; and
   iii. a computing platform in communication with said camera, adapted for: analyzing said eye movement data; and determining, based on said eye movement data, a familiarity category selected from multiple familiarity categories, wherein said familiarity category defines a familiarity of said subject with said stimulus
b. a lying detection system LDS for implementing a lying detection technique on said subject
   wherein said SSF and said LDS are operationally linked such that the output of said SSF may be combined with the output of said LDS to obtain a detection quality result with a more than additive accuracy of detection of symptomatic behavior of lying relative to the accuracy of detection of same obtained from either determining a subject's familiarity with given stimuli or implementing a lying detection technique on said subject and obtaining a lying detection result therefrom alone.

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a system and method for determining familiarity with stimuli.

In the following detailed description, numerous specific details are set forth in order to provide a thorough understanding of embodiments of the present invention. However, those skilled in the art will understand that such embodiments may be practiced without these specific details. Reference throughout this specification to “one embodiment” or “an embodiment” means that a particular feature, structure, or characteristic described in connection with the embodiment is included in at least one embodiment of the invention.

The term “SSF” hereinafter refers to a system for determining a subject's familiarity.

The term “LDS” hereinafter refers to a lying detection system of any kind. It is a core pupose of the present invention to provide embodiments wherein both the SSF and the LDS are integrated into a system for detecting symptomatic behavior of lying, which, by combining the results of both systems, provide an unexpectedly reliable or accurate detection result. This synergistic effect will be highly useful and is very much in demand.

The term “detection quality result” refers to the reliability or strength or certainty of conclusions or calculations or results concerning a subject who has been subjected to the abovementioned system for determining a subject's familiarity or a lying detection system of any kind or a combination of both, either in series or in parallel or simultaneously or contemporaneously.

The term ‘admitted familiarity’ hereinafter refers to the class of information that is familiar to a subject, and that he admits to be familiar to him.

The term ‘admitted unfamiliarity’ hereinafter refers to the class of information that is unfamiliar to a subject, and that he admits to be unfamiliar to him.

The term ‘denied familiarity’ hereinafter refers to the class of information that is known to a subject and that the subject denies to be familiar to him.

The term ‘denied unfamiliarity’ hereinafter refers to the class of information that is unfamiliar to a subject, and that the subject denies to be unfamiliar (or claims to be familiar) to him.

The term ‘claimed familiarity’ hereinafter refers to the class of information that is unfamiliar to a subject, and that the subject denies to be unfamiliar (or claims to be familiar) to him.

The term ‘training data’ hereinafter refers to data used for purposes of ‘teaching’ an adaptive algorithm such as the support-vector machine (SVM), neural network, or the like. Training data generally consist of examples where the correct ‘answer’ such as class or score is known, and are used to better the performance of such algorithms using known methods such as backpropagation.

The term ‘plurality’ refers hereinafter to any positive integer greater than 1, e.g., 2, 5, or 10.

It is within the scope of the present invention to provide methods for detecting symptomatic behaviours of lying.

Prior art methods of lie detection, as previously noted, have their drawbacks and are often unreliable. The subject is placed under psychological stress of one type or another, which is translated into detectable physiological effects, which may sometimes be circumvented, falsified or masked. The present application provides systems and methods for measuring familiarity and combining the obtained familiarity results with lie detection results, thereby obtaining a greater than additive accuracy or strength of result than if either method was used seperately.

Thus the idea is to augment the eye tracking techniques for knowing if a person is familiar with a picture. The augmentation may be done by adding one or more techniques for detecting if a person is lying. Each of which gives some quality measure. Adding or combining these measurements together will increase the overall detection quality.

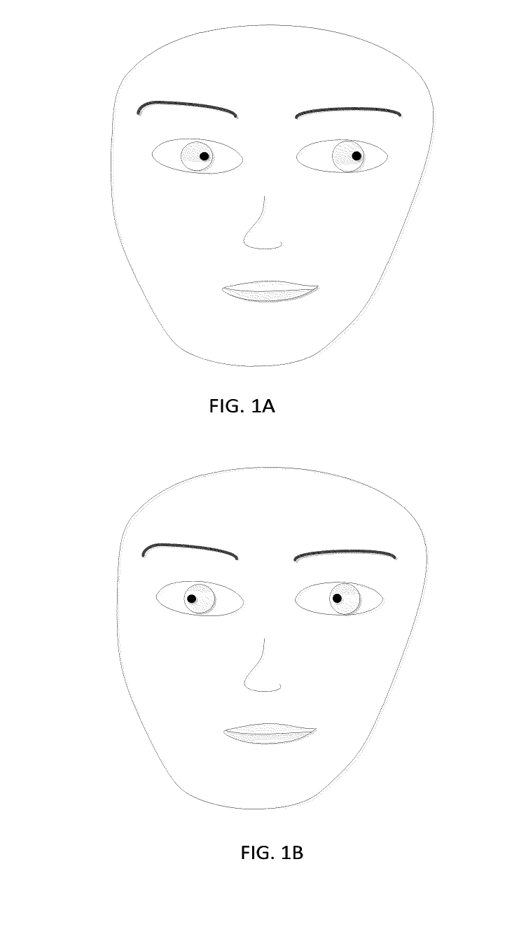

As detailed in the background section it appears that use of eye movement data may allow an effective system of mental state determination, for purposes such as lie detection, comprehension testing, and the like. Ideally such a system will be free from operator bias and therefore will be as automated as possible, e.g. by use of computerized analysis of results as opposed to human analysis. A simple example of such analysis is shown in FIGS. 1A, 1B wherein gazing to the left (FIG. 1B) correlates with creative thinking, while gazing to the right (FIG. 1A) correlates with recall of stored memory. Note that this example is not necessarily correct to any great accuracy but simply an illustration of a commonly held belief concerning a correlation between eye movement and mentation.

In light of the above research, a method and system is herein provided to detect whether a person is concealing knowledge and/or pretends to possess knowledge he does not really have. Such a system is applicable to a wide span of applications. To name a few: when selecting terror suspects in an airport or when trying to verify that a candidates' qualification are genuine.

The method and associated system might be considered similar to a lie detector in its intent. However unlike a lie detector that requires special physical preparation and physical attachments, the current system is non-intrusive and strives to be transparent. Another important advantage is high reliability of results.

In a preferred embodiment of the invention a person is asked to look at a series of pictures including images expected to be: 1) familiar images; 2) images expected to be unfamiliar; 3) images suspected to be familiar despite claims of ignorance; and 4) images suspected to be unfamiliar despite claimed expertise. In keeping with the definitions listed above these categories will hereinafter be referred to respectively as: admitted familiar, admitted unfamiliar, denied familiar, and denied unfamiliar.

Some examples of images that might be shown to a suspected terrorist bomb maker:
Apples and Oranges (admitted familiar)
A scheme of an explosive device (denied familiar)
A picture of a terrorist (denied familiar)
An SEM image of a micro-organism (admitted unfamiliar)

While the suspect studies the pictures, he may be guided to look again at the same ones after being given information about them. In other cases, he might be asked to repeat a few pictures, after intermediate analysis is inconclusive. Throughout the session, the suspect's eye movements are recorded for analysis or analyzed in real time. The entire session, including what pictures are to be shown and what is said to the suspect at what time, are pre-planned by the tester and/or by the testing algorithm.

The analysis of the movement may be accomplished, inter alia, by a so-called “classification” algorithm. One skilled in the art will recognize the variety and precision of machine learning techniques that classify data into categories. Computer programs and algorithms have been devised to train on example data and then successfully classify new data. In the case of the current invention one or more of these algorithms are trained using a training set that includes sampled eye movements of different people reflecting the four types of classes described above. Alternatively the training set may be specific to a certain person, or specific to a certain subset of the population such as Caucasian males, French females, and the like. In any case the training set will generally consist of examples of one or more of the classes of interest, namely admitted familiar, admitted unfamiliar, denied familiar, and denied unfamiliar. Examples of such algorithms include neural nets, the support vector machine [SVM] Decision Trees, Bayesian Networks and a host of others. It is also within provision of the current invention that the training set for any of these algorithms may be modified or rebuilt entirely by new eye movement data from a given subject. This will allow for the possibility of large variability between subjects and may conceivably increase the accuracy of the results; in effect the system learns to classify responses of a subject on an individual basis.

As will be clear to one skilled in the art, for some of the aforementioned algorithms there will be a necessary period of training during which training data must be used to ‘teach’ the algorithm by way of example. For example in the SVM and neural nets, the algorithms are initially given training data along with the correct classifications thereof. Thus examples of eye-movement data from each of the four categories (admitted familiar, admitted unfamiliar, denied familiar, denied unfamiliar) would be provided to the algorithm in this stage. These examples must be known to fall into one of the categories in order to correctly train the algorithm.

It will be obvious to one skilled in the art that certain variations on this method are possible. For example, instead of strict membership in one class of a set of classes, degree of membership in each of a set of classes may be determined. Alternatively, other forms of quantitative measurement may be provided, such as ratings on different physical or psychological scales. Furthermore the particular classes mentioned can be replaced by other classes if found to be more suitable to a particular task.

After the program is trained and its classification ability is established as statistically significant, it is used to classify unknown data, identifying the class that a subject belongs to.

The system of the current invention includes the following equipment:

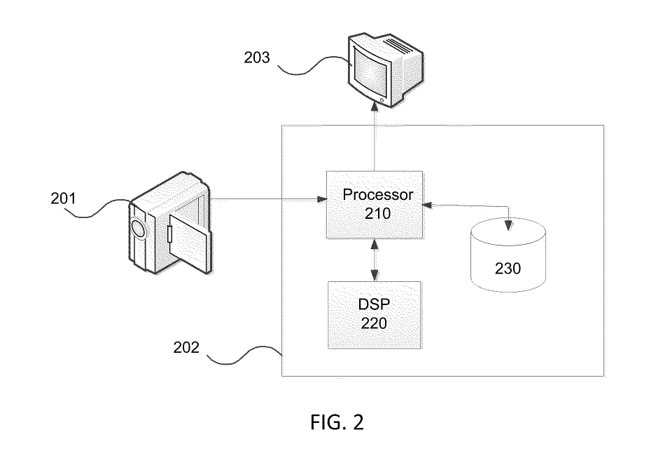

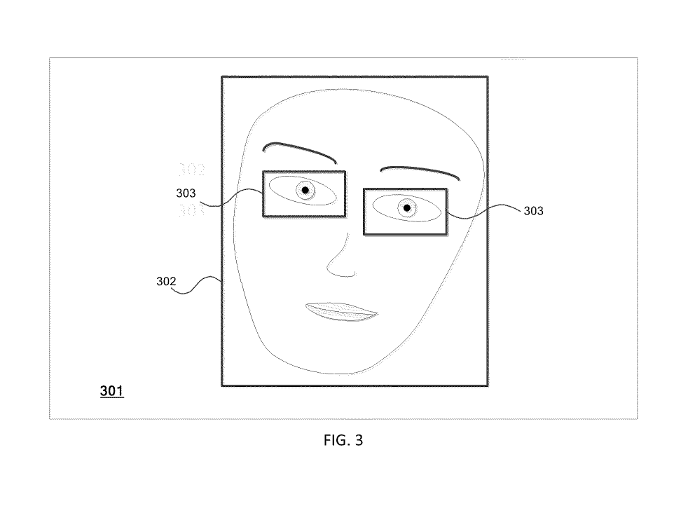

A camera is provided to capture eye movement data. For example, the ASL 504 eye movement detection camera which was used in the scientific experiments referred to in the background may be used. The camera may optionally be positioned in a concealed manner. In FIG. 2 an eye-movement tracking camera 201 is shown along with some dedicated hardware 202 adapted to convert the raw data from the camera into eye-movement data such as gaze direction. This hardware may for instance take the image 301 shown in FIG. 3, and provide, amongst others, outputs of face position 303 and eye position 302.

Dedicated hardware 202 includes: a processor 210, coupled to an optional digital signal processor (DSP) 220 and to a storage device 230. Storage device 230 stores stimuli, eye movement images (or video) and eye movement data. DSP 220 is configured to: convert eye movement images into eye movement data; identify face position 303 and eye position 302 by using algorithms such as, for example: pattern recognition, morphological image processing and the like; and classify eye movement data into multiple familiarity categories. Processor 210 is configured to conduct the presentation of the stimuli and to control storage device 230 and DSP 220. Processor 210 is coupled to camera 201 and to a display 203 for displaying the stimuli.

According to an embodiment of the invention, both the functionality of DSP 220 and the functionality of processor 210 can be implemented by processor 210. According to another embodiment, DSP 220 and processor 210 are enclosed in two separated computing platforms. A first computing platform, which includes DSP 220, is configured to interpret eye movement images and eye movement data and to classify the eye movement data. The first computing platform is coupled to camera 201. A second computing platform is configured to conduct a stimuli presentation and is coupled to display 203. A computer desktop or laptop is provided, that records the digital signals that the camera outputs. In some embodiments of the invention another computer is used to generate the visual test by means of software controlling the sequence, duration and type of images projected to the suspect. An alternative is that the projection and the analysis will be performed on separate machines. In this latter case both computers need not be at the same location.

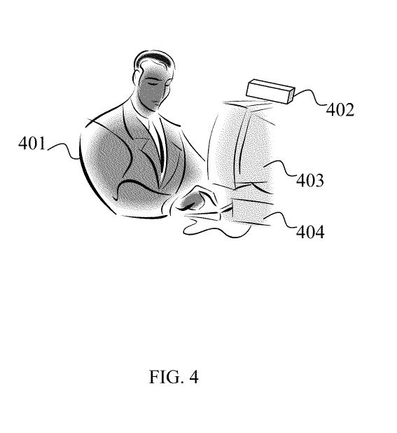

The system may appear as in FIG. 4, where the subject 401 sits before a standard computer screen 403 that is provided with eye-tracking camera 402. The eye movement data recorded by the camera 402 is analyzed by the computer 404 in light of the visual stimuli presented by computer 404 on screen 403.

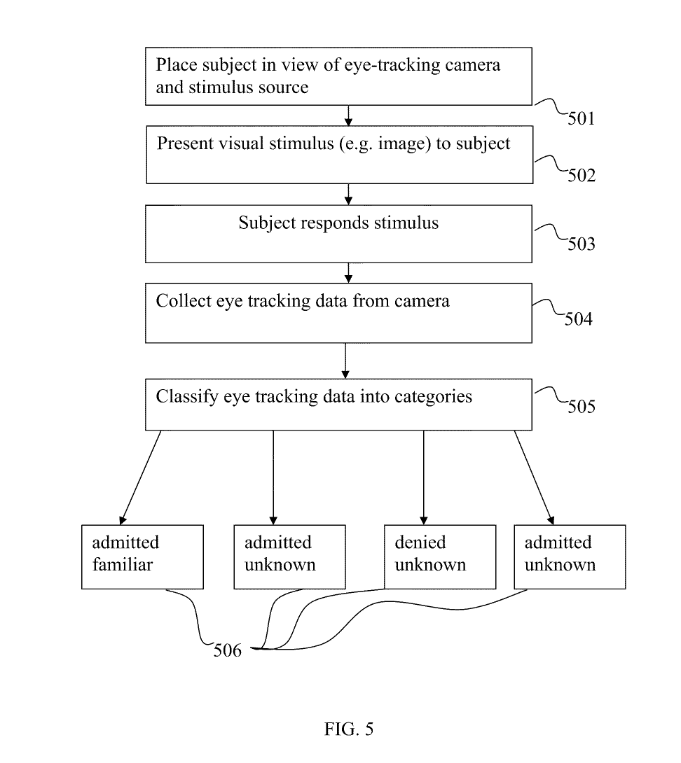

The method is shown in brief outline in FIG. 5. The subject is first placed where he can be presented with stimuli and his eyes can be observed by the tracking camera, in step 501. Then visual stimuli such as images are presented, in step 502. Then the subject responds to the stimulus, such as by describing the image or simply observing it, in step 503. During this response period eye tracking data is recorded, in step 504.

After the eye tracking data has been collected, it is classified into categories in step 505, in one embodiment this being into categories ‘admitted familiar’, ‘admitted unfamiliar’, ‘denied familiar’, ‘denied unfamiliar’.

DSP 220 executes an analysis algorithm that can be trained to classify different data generated by the eye movement detector.

According to an embodiment of the invention, step 505 may utilize a familiarity indicative algorithm for interpreting eye movements indicative of familiarity. The familiarity indicative algorithm concentrates on eye fixations that are captured and measured during a presentation of a specific visual stimulus (e.g. a specific image). The term ‘fixation’ refers to focusing on a specific spot of the visual stimulus. The familiarity indicative algorithm may measure the number of fixations, the position of the fixations, the density of the fixations, i.e. their spatial distribution, the duration of the fixations, and so on.

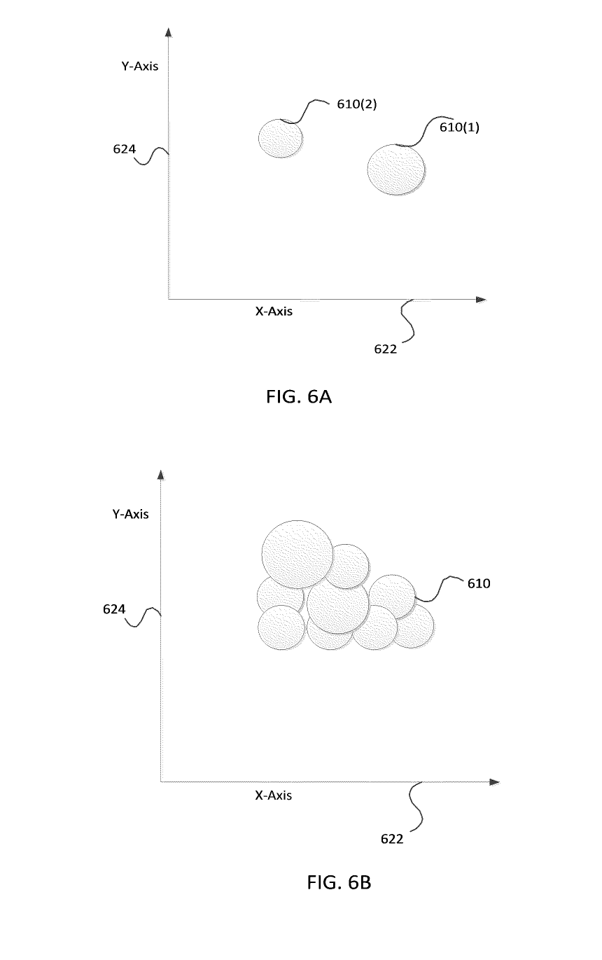

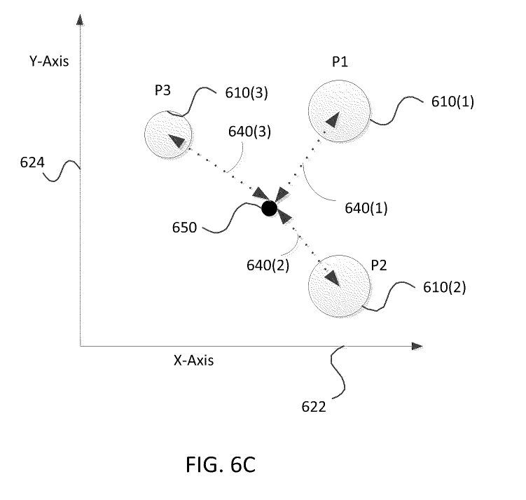

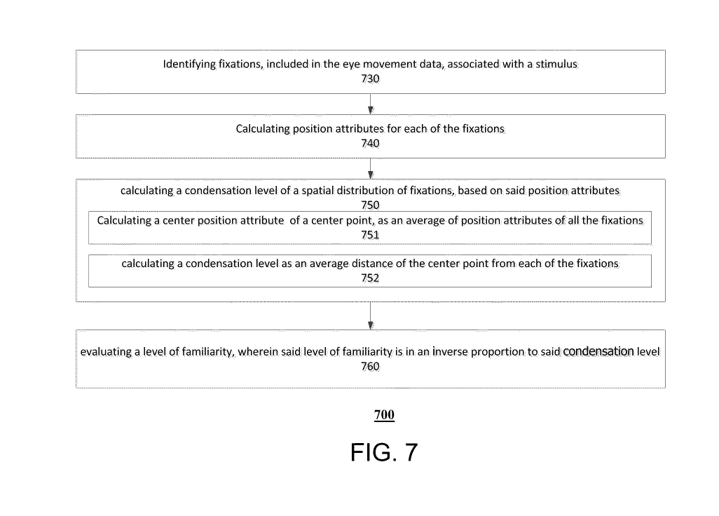

FIG. 6A illustrates a graph of a spatial distribution of fixations that were recorded as part of eye movement data, in response to a stimulus, that is familiar to the subject. X-axis 622 and Y-axis 624 defines a coordinates system of a visual stimulus, which is the range where the fixations are expected to be measured. Two distinct fixations 610(1) and 610(2) are shown.

FIG. 6B illustrates a graph of a spatial distribution of fixations that were recorded as part of eye movement data of another subject that is not familiar with the same stimulus. The fixations, collectively denoted as 610, are comparatively condensed.

Referring to FIG. 6C, a position (e.g. coordinates) of a center point 650 is calculated. Center point 650 is the center of all fixations 610 that were recorded during a stimulus presentation.

An X-coordinate (X_{center}) of center point 650 is the average of X-coordinates (Xᵢ) of all fixations 610 (in FIGS. 6C, 610(1), 610(2) and 610(3)), according to the formula:

$${X}_{center} = ( {\sum }_{i = 1}^{n} {X}_{i} ) / n$$

Wherein n is the number of fixations that were recorded e.g., n=3 in FIG. 6C.

A Y-coordinate (Y_{center}) of center point 650 is the average of Y-coordinates (Yᵢ) of all fixations:

$${Y}_{center} = ( {\sum }_{i = 1}^{n} {Y}_{i} ) / n$$

A distance from center point 650 is calculated for each fixation 610: distance 640(1), denoted by a doted line, is the distance between center point 650 and fixation P1 610(1), distance 640(2) is the distance between center point 650 and fixation P2 610(2) and distance 640(3) is the distance between center point 650 and fixation P3 610(3).

A condensation level of a spatial distribution of the fixations can be defined by an average distance of all fixations 610 from center point 650, as described by the expression:

$$( {\sum }_{i = 1}^{n} Distance ( {P}_{i} ( {X}_{i} , {Y}_{i )} , {P}_{center} ( {X}_{center} , {Y}_{center} ) ) / n$$

Pi (Xi,Yi) —represents the location of fixations P1 610(1), P2 610(2) and P3 610(3), n in this case is 3.

Center point 650 is also known to be the center of mass of these fixations. The average distance of the fixations from this center of mass represents the condensation of the fixations.

The inventors have executed a vast number of experiments and have found that a lower level of condensation is typically calculated for fixations of a subject who is unfamiliar with the stimulus and vice versa.

FIG. 7 illustrates a method 700 for evaluating a familiarity level. Method 700 may be part of stage 505 of FIG. 5.

Method 700 starts with a stage 730 of identifying fixations, included in eye movement data, associated with a stimulus.

Stage 730 is followed by a stage 740 of calculating position attributes for each of the fixations. The position attributes may be coordinates, e.g., X-Y coordinates, of a fixation within a plane of an image stimulus. The plane of the image may be mapped into an X-Y coordinate system, wherein the bottom left corner of the image is defined as (x=0, y=0).

Stage 740 is followed by a stage 750 of calculating a condensation level of a spatial distribution of the fixations, based on the position attributes. Stage 750 may include a stage 751 of calculating a center position attribute of a center point, as an average of position attributes of all the fixations. Stage 750 may further include a stage 752 of calculating a condensation level as an average of the distances from the center point to each of the fixations. The calculation is based on the position attributes of all the fixations and the center position attribute.

Stage 750 is followed by a stage 760 of evaluating a level of familiarity, wherein the level of familiarity is in a direct proportion to said condensation level, i.e. a lower level of familiarity will be evaluated for a low condensation level and vice versa.

According to an embodiment of the invention, a combination lying machine is provided. The lying machine combines two techniques: (i) the eye tracking technique, described above, for detecting a familiarity of a subject with a stimulus; and (ii) at least one lying detection technique for evaluating authenticity of answers given by the subject.

The combination lying machine includes all the elements of dedicated hardware 202 in addition to psychophysiological sensors known in the art.

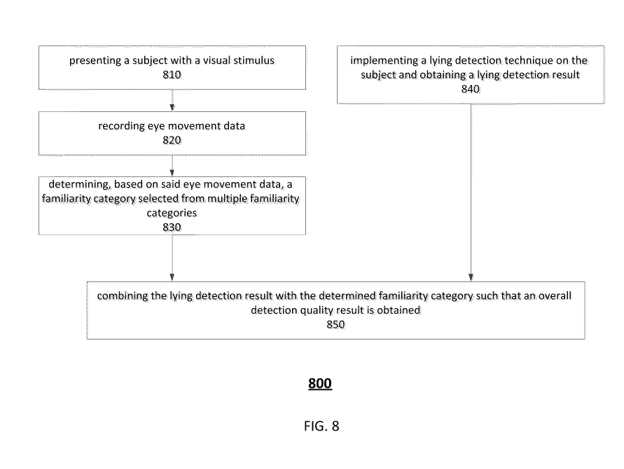

FIG. 8 illustrates a flowchart of a method 800 for detecting a symptomatic behavior of lying. The method includes: a stage 810 for presenting a subject with a visual stimulus. A stage 820, for recording eye movement data, is executed concurrently with step 810. Step 820 is followed by a stage 830 of determining, based on said eye movement data, a familiarity category selected from multiple familiarity categories. The familiarity category defines a familiarity of the subject with the visual stimulus.

Method 800 also includes a stage 840 for implementing a lying detection technique on the subject and obtaining a lying detection result. Stage 840 may include posing a question to the subject and reading at least one physiological measurement during a response of the subject to the question. The reading of the physiological measurement utilizes at least one physiologic sensor. Stage 840 may be performed before, after or during stages 810-830.

Stages 830 and 840 are followed by a stage 850 of combining the lying detection result with the determined familiarity category such that an overall detection quality result is obtained. The overall detection quality can be measured by a percentage value that represents the statistical accuracy of the overall detection quality result. The percentage value is higher than a second percentage value that represents a statistical accuracy of a regular lying test result.

A User Interface is provided for controlling the test and indicating the class which the system believes the suspect belongs to.

The voice directing the suspect during the test may be generated by the computer as well, or by a human specialist interrogating him, or by another source, for example a database of recorded voice samples.

In some embodiments of the invention the system is manned by an interrogator, while in other embodiments no interrogator is present.

It may be found preferable not to disclose the purpose of the tests of the current invention, and/or to hide the existence of the system altogether (for example by concealing the eye movement camera in a wall of an interrogation room, or by performing the eye movement analysis on video data recorded from a normal video camera, or the like).

Useful features of the current invention include the facts that it is non intrusive, accurate, objective (automated), and can be implemented in an undetected manner thus avoiding any possible countermeasures.

Some examples of use of the system are given below.

### Example 1

### Screening a Candidate for a Sensitive Job

In this scenario, a factory is suspicious of a candidate who enlists to fill a cleaning man's job. The factory fears that he has been recruited to commit commercial espionage for the competition. In such a case, he will be concealing prior knowledge about the competitive company, or about critical technical processes. He could be debriefed about such by his senders in the following way.

The candidate will be shown different pictures while employing the eye-movement analysis method of the current invention. Some would be innocent images unrelated to the suspicion of espionage, but others will contain trade secrets concerning the company's business about which the candidate is not supposed to be knowledgeable. Other pictures might be of managers in the competing company—in an effort to determine whether the managers are familiar to the candidate. Others may contain words in the language of the competitor (for example in French) again, in an effort to establish if they are unfamiliar to him.

If the system's analysis indicates that the candidate is knowledgeable about subjects he has claimed not to be, the company may decide to further investigate him or simply not to hire.

In this example we have illustrated the range of prior knowledge that may be determined using the system, including knowledge of people, processes, and languages. It should be stressed that the type of knowledge that can be verified or falsified using the system is not limited to this small group but rather encompasses the full range of human knowledge.

### Example 2

### Screening a Potential Candidate for a High Tech Job

In this scenario, a recruiting company would like to prove that a candidate indeed has the qualifications he pretends to have. Suppose that a person who claims to have a PhD in molecular biology is being interviewed for a job in a high tech firm. The candidate has presented a CV claiming knowledge in the domain of certain complex proteins.

The interviewers (or computer code), using the system of the current invention, would present him with a series of pictures, and ask him to explain what he sees. Some pictures might contain simple questions, such as to describe what he sees while looking at images of DNA building blocks. Other images however could depict complex proteins which would require a higher level of understanding to describe. When presented with familiar images, the eye movements of the viewer will be qualitatively different from his eye movements when presented with unfamiliar images. The classification algorithm trained to detect these differences can then classify a given set of eye movements into one of the four categories described above, in this case either finding his responses to be either admitted familiar or denied unfamiliar.

Based upon the results of the candidate's eye movement analysis, the person may be determined competent enough and qualified for an expert interview, or rejected.

### Example 3

### Screening a Person in the Airport in Search of Terrorists

The current invention offers a cheap and efficient way of screening travelers at an airport, seaport, or other travel gateway. Often, security and police may have prior knowledge about a terror act that may be in the making. A suspect is isolated and presented with the prepared image test of the current invention. Pictures of members of terrorist organization (based on prior intelligence) are planted in between pictures of known-to-be unfamiliar and known-to-be familiar faces. Sporadic diagrams of explosive devices and common terrorist weapons may also be displayed. Inscriptions in the language and religion of the suspected terrorists may be shown as well. Pictures of landscapes, places, or characters from the perpetrators origin may be displayed. The suspects eye movements are detected and classified for each image presented, falling into the categories of the system, namely admitted familiar, admitted unfamiliar, denied familiar, and denied unfamiliar. (The category of denied unfamiliar may be generated for instance by producing an image of a city or neighborhood that the suspected terrorist claims to have visited relatives in before.) Based on the results of the system analysis, the suspect would be either released or detained for further interrogation.

It is within provision of the invention that the categories mentioned above be generalized or modified, for example by using categories {‘lying’, ‘telling truth’}, or categories {‘completely familiar’, ‘passing familiarity’, ‘expert knowledge’}, categories including emotional states such as {‘nervous but not hiding knowledge’, ‘nervous and hiding knowledge’, ‘not nervous and not hiding knowledge’, ‘not nervous and hiding knowledge’}, and the like.

It is within provision of the invention that the classification algorithm mentioned above be replaced by another computerized algorithm, such as an expert system, pattern matching algorithm, heuristic algorithm, and others which will be obvious to one skilled in the art.

It is within provision of the invention that the images presented by the system include people, places, things, texts, moving images (videos), test patterns, and three-dimensional images.

It is within provision of the invention that the stimuli presented to the subject not be limited to visual information, but rather may include auditory stimulation, presentation with actual objects or people, and other sensory input including taste, smell, and touch. Furthermore combinations may be used, for example images and sounds.

It is within provision of the invention that information gathered by the system concerning the eye movements of the subject include: gaze direction, fixation duration, saccade duration, saccade velocity, head position, head velocity, and the like as will be obvious to one skilled in the art.

It is within provision of the invention that it be used in suspect identification, such as in a police lineup. In this case two parties might be subject to analysis by the system, namely the suspect, and a complainant or alleged witness.

Another example of the use of the system would be for in identifying criminal activity by means of judging familiarity with a crime or crime scene, for example familiarity with the interior of a particular house, or familiarity with the appearance of a murder victim.

A method for judging familiarity with a person or object may be applied where a person or object suspected to be familiar to a subject is placed in an image with a group of other people or objects; in the analysis of such situations, it may be found, for instance, that familiar objects/people enjoy greater visual attention than unfamiliar objects/people, or the reverse. It will be appreciated by one skilled in the art that since such situations may be analyzed and ‘learned’ by various algorithms like the support vector machine, detailed research knowledge concerning these types of correlations are not absolutely necessary.

The eye-tracking system and method of the current invention can be utilized to judge advertising effectiveness; for example, webcams, surveillance cameras, or cameras hidden in billboards or near video screens may be used to record viewer attention data. This data may prove of great worth to advertising firms, who will be able to determine advertising effectiveness and/or attention information concerning commercials, billboards, video ads, web banners, and the like.

It is within provision of the invention that the eye data recording system may be a dedicated piece of hardware in communication with the eye-tracking camera, instead of residing in a standard computer.

It is within provision of the invention that images be presented by means of a projector, video screen, or by way of printed photographs.

It is within provision of the invention that the eye-tracking camera used be a dedicated eye-tracking camera, or another video-capable device provided with post processing means to determine the relevant gaze direction parameters. For example, it may be found that in certain cases a standard webcam and image processing algorithms suffice to determine gaze direction and associated data with sufficient precision.

It is within provision of the invention that results be presented to the system operator in terms of stimulus-class pairs (which stimuli are found to be associated with which class (admitted known, admitted unknown, etc.)), optionally with some indication of the degree of confidence in a given classification. It is within provision of the invention that certain images or stimuli or transformations thereof be repeated, in order to increase the confidence in classification.

As will be clear to one skilled in the art, a skilled interrogator may increase the effectiveness of the system by psychological means.

It is within provision of the invention that various transformations of stimuli be applied, such as turning a figure upside-down or otherwise rotating it, inverting it left-right or up-down, reversing the time sequence of a video, changing colors of an image, or other transformations as will be known to one skilled in the art.

It should be emphasized that the stimuli of the invention need not be images, but can also comprise text. The system may be used to judge comprehension level, comprehension speed, and familiarity with a given word, body of text, language, concept, or the like.

It is within provision of the invention that analysis be carried out on video data in real time, or that such data be collected, recorded, and processed at a later time. Alternatively the video data may be analyzed and processed, then stored.

1. A method for determining a subject's familiarity with given stimuli comprising steps of:
a. providing an eye-movement detection camera adapted to capture and record eye movement data of said subject;
b. providing a display means, adapted for presentation of said stimuli to said subject,
c. providing a computing platform in communication with said camera, adapted for analyzing said eye movement data;
d. presenting said subject with a stimulus;
e. recording eye movements data associated with said subject's response to said stimulus, by said eye-movement detection camera and said computing platform; and
f. determining, based on said eye movement data, a familiarity category selected from multiple familiarity categories, wherein said familiarity category defines a familiarity of said subject with said stimulus.

2. The method of claim 1, wherein said platform adapted for presentation of stimuli is the same computing platform adapted for determination of said familiarity category.

3. The method of claim 1, wherein said multiple familiarity categories include: admitted familiarity, admitted unfamiliarity, denied familiarity, and denied unfamiliarity.

4. The method of claim 1, wherein said determination of said familiarity category is accomplished by means of an algorithm selected from a group consisting of: support vector machine [SVM], decision tree, Bayesian network, neural network, genetic algorithm, expert system, pattern matching algorithm, heuristic algorithm, or combinations thereof.

5. The method of claim 4, wherein said algorithm is trained on training data selected from a group consisting of: data gleaned from the population at large; data gleaned from population subsets; and data gleaned from said subject.

6. The method of claim 1, wherein said eye movement data is selected from a group consisting of: gaze direction, fixation duration, saccade duration, saccade velocity, head position, head velocity, or combinations thereof.

7. The method of claim 1, wherein said stimuli are selected from a group consisting of: images known to be familiar to said subject, images known to be unfamiliar to said subject, images suspected to be familiar to said subject, images suspected to be unfamiliar to said subject, images of persons, images of places, images of things, videos, digital media, persons, objects, auditory information, tactile stimuli, olfactory stimuli, or combinations thereof.

8. The method of claim 1, further requesting a response from said subject to said stimuli, selected from a group consisting of: talking about said stimuli, observing said stimuli, writing about said stimuli, or classifying said stimuli.

9. The method of claim 1, wherein said display means is selected from a group consisting of: a computer display, projector, photograph, sketch, or drawing.

10. A system for determining a subject's familiarity with given stimuli consisting of:
a. display means adapted for presentation of said stimuli to said subject,
b. an eye-movement detection camera adapted to capture and record eye movement data, associated with said subject's response to a stimulus; and
c. a computing platform in communication with said camera, adapted for: analyzing said eye movement data; and determining, based on said eye movement data, a familiarity category selected from multiple familiarity categories, wherein said familiarity category defines a familiarity of said subject with said stimulus

11. The system of claim 10, wherein said multiple familiarity categories include: admitted familiarity, admitted unfamiliarity, denied familiarity, or denied unfamiliarity.

12. The system of claim 10, wherein said determination of a familiarity category is accomplished by means of an algorithm selected from a group consisting of: support vector machine [SVM], decision tree, Bayesian network, neural network, genetic algorithm, expert system, pattern matching algorithm, heuristic algorithms, or combinations thereof.

13. The method of claim 12, wherein said algorithm is trained on training data selected from a group consisting of: data gleaned from the population at large; data gleaned from population subsets; or data gleaned from said subject.

14. The system of claim 10, wherein said eye movement data is selected from a group consisting of: gaze direction, fixation duration, saccade duration, saccade velocity, head position, head velocity, or combinations thereof.

15. The system of claim 10, wherein said stimuli are selected from a group consisting of: images known to be familiar to said subject, images known to be unfamiliar to said subject, images suspected to be familiar to said subject, images suspected to be unfamiliar to said subject, images of persons, images of places, images of things, videos, digital media, persons, objects, auditory information, tactile stimulation, olfactory stimulation, or combinations thereof.

16. The system of claim 10, further requesting a response from said subject to said stimuli, selected from a group consisting of: talking about said stimuli, observing said stimuli, writing about said stimuli, or classifying said stimuli.

17. The system of claim 10, wherein said display means is selected from a group consisting of: a computer display, projector, photograph, sketch, or drawing.

18. The method of claim 1, wherein said eye movement data comprises position attributes of fixations, and wherein said determining of said familiarity category is based on said position attributes.

19. The method of claim 18, wherein the step of determining comprises:
calculating a condensation level of a spatial distribution of said fixations, based on said position attributes; and
evaluating a level of familiarity, wherein said level of familiarity is in a direct proportion to said condensation level.

20. A Method for detecting symptomatic behavior of lying comprising steps of:
a. determining a subject's familiarity with given stimuli comprising steps of
i. providing an eye-movement detection camera adapted to capture and record eye movement data of said subject;
ii. providing a display means, adapted for presentation of said stimuli to said subject,
iii. providing a computing platform in communication with said camera, adapted for analyzing said eye movement data;
iv. presenting said subject with a stimulus;
v. recording eye movements data associated with said subject's response to said stimulus, by said eye-movement detection camera and said computing platform; and
vi. determining, based on said eye movement data, a familiarity category selected from multiple familiarity categories, wherein said familiarity category defines a familiarity of said subject with said stimulus and
b. implementing a lying detection technique on said subject and obtaining a lying detection result therefrom
c. combining said lying detection result with said determined familiarity category such that an overall detection quality result is obtained.

21. The method according to claim 20 wherein said detection quality result has a more than additive accuracy of detection relative to the accuracy of detection obtained from either determining a subject's familiarity with given stimuli or implementing a lying detection technique on said subject and obtaining a lying detection result therefrom alone.

22. A system for detecting symptomatic behavior of lying comprising
a. a system for determining a subject's familiarity SSF with given stimuli consisting of:
i. display means adapted for presentation of said stimuli to said subject,
ii. an eye-movement detection camera adapted to capture and record eye movement data, associated with said subject's response to a stimulus; and
iii. a computing platform in communication with said camera, adapted for: analyzing said eye movement data; and determining, based on said eye movement data, a familiarity category selected from multiple familiarity categories, wherein said familiarity category defines a familiarity of said subject with said stimulus
b. a lying detection system LDS for implementing a lying detection technique on said subject
wherein said SSF and said LDS are operationally linked such that the output of said SSF may be combined with the output of said LDS to obtain a detection quality result with a more than additive accuracy of detection of symptomatic behavior of lying relative to the accuracy of detection of same obtained from either determining a subject's familiarity with given stimuli or implementing a lying detection technique on said subject and obtaining a lying detection result therefrom alone.
